SEQUENCE LISTING (2 sequences)

SEQ ID NO: 1 - protein, 40 aa, Artificial Sequence
  synthetic
YXEGTFTSDYSIYLDKQAAXEFVNWLLAGGPSSGAPPPSC

SEQ ID NO: 2 - protein, 40 aa, Artificial Sequence
  synthetic
YXEGTFTSDYSIYLDKQAAXEFVNWLLAGGPSSGAPPPSK

(19) United States
(12) Patent Application Publication
(10) Pub. No.: US 2017/0112897 A1
(43) Pub. Date: Apr. 27, 2017

(54) METHODS FOR TREATING BRAIN INSULIN RESISTANCE

(71) Applicants: Cedars-Sinai Medical Center, Los Angeles, CA (US); Research Foundation of The City University of New York, New York, NY (US)

(72) Inventors: Konrad Talbot, Los Angeles, CA (US); Hoau-Yan Wang, Philadelphia, PA (US); Keith L. Black, Los Angeles, CA (US)

(73) Assignees: Cedars-Sinai Medical Center, Los Angeles, CA (US); Research Foundation of The City University of New York, New York, NY (US)

(21) Appl. No.: 15/332,880

(22) Filed: Oct. 24, 2016

Related U.S. Application Data
(60) Provisional application No. 62/245,895, filed on Oct. 23, 2015.

(51) Int. Cl. A61K 38/16; A61K 31/13; A61K 45/06

(52) U.S. Cl. CPC A61K 38/16; A61K 45/06; A61K 31/13

(57) ABSTRACT

The invention provides methods for treating brain insulin resistance and/or diseases associated therewith in a subject in need thereof. The methods include providing a composition comprising a dual GLP-1 receptor/GIP receptor (GLP-1R/GIPR) agonist and administering an effective amount of the composition to the subject to treat brain insulin resistance and diseases associated therewith.

### CROSS-REFERENCE TO RELATED APPLICATIONS

This application claims the benefit of priority under 35 U.S.C. §119(e) to U.S. Provisional Application No. 62/245,895, filed on Oct. 23, 2015, the contents of which are herein incorporated by reference in its entirety.

### FIELD OF INVENTION

Embodiments of the invention provide methods for treating diseases associated with brain insulin resistance in subject in need thereof, using dual glucagon-like peptide 1 (GLP-1)/gastric inhibitory peptide (GIP) receptor agonists.

### BACKGROUND

All publications cited herein are incorporated by reference in their entirety to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Alzheimer's disease (AD) poses one of the great challenges to 21^{st} century medicine. It leads to a state of dementia that robs those afflicted of their ability to remember, think, and communicate with others and is the 6th leading cause of death in the U.S. More than century after its discovery, however, there are no clinically effective treatments for AD that substantially slow or stop its progression. If this situation persists, it is estimated that the number of AD dementia cases in the U.S. will rise from 5.2 million today to 13.8 million by 2050 with annual health care costs for those individuals rising from $136 billion today to $660 billion in today's dollars. These costs are derived from estimates of health care costs for all dementia cases, about 60% of which are AD cases.

There is consequently an urgent need for discovery of treatments markedly slowing AD progression. Indeed, it has been estimated that disease-modifying treatments delaying onset of AD dementia by just 5 years could reduce the number of cases by 57% and reduce Medicare costs by 45%. Provided herein are methods using a dual incretin receptor agonist (dual IRA) to treat brain insulin resistance, which is now recognized as a major pathogenic factor in AD.

### SUMMARY

Provided herein is a method for treating, inhibiting, reducing the severity of and/or slowing progression of a disease-state in a subject in need thereof comprising, consisting of or consisting essentially of providing a dual IRA and administering a therapeutically effective amount of the dual IRA to the subject so as to treat, inhibit, reduce the severity of and/or slow progression of the disease-state in the subject. In one embodiment, the subject has diabetes. In another embodiment, the subject does not have diabetes.

In one embodiment, the disease-state is brain insulin resistance. In an embodiment, the disease-state is a disease associated with brain insulin resistance. In another embodiment, the disease-state is mild cognitive impairment (MCI). In a further embodiment, the disease-state is Alzheimer's disease (AD) dementia.

In some embodiments, the incretin receptor agonist activates the GLP-1 receptor and the GIP receptor and is thus known as a dual GLP-1R/GIPR receptor agonist (i.e., a dual IRA).

In one embodiment, the dual GLP-1/GIP receptor agonist is an unmodified peptide comprising, consisting of or consisting essentially of the sequence YXEGTFTSDYSIYLDKQAAXEFVNWLLAGGPSSGAPPPSC-NH₂ (SEQ ID NO.: 1), wherein X is aminoisobutyric acid, (for example, peptide 17 in Finan, B. et al. Science Translational Medicine 5: 209ra151, 2013). In some embodiments, the peptide having SEQ ID NO: 1 is acylated or PEGylated.

In another embodiment, the dual GLP-1/GIP receptor agonist is a peptide comprising, consisting of or consisting essentially of the sequence YXEGTFTSDYSIYLDKQAAXEFVNWLLAGGPSSGAPPPSK (SEQ ID NO.: 2), (for example, peptide 18 in Finan, B. et al. Science Translational Medicine 5: 209ra151, 2013), wherein X is aminoisobutyric acid. In some embodiments, the peptides having the sequence of SEQ ID NO: 2 have been acylated or PEGylated. In one such embodiment, the peptide having SEQ ID NO: 2 is acylated at Lys-40 ((K⁴⁰-C₁₆ acyl)-NH₂), (for example, compound 19 in Finan, B. et al. Science Translational Medicine 5: 209ra151, 2013).

In a further embodiment, the peptide having SEQ ID NO:2 is PEGylated at Cys-24 (for example, compound 21 in Finan, B. et al. Science Translational Medicine 5: 209ra151, 2013).

In another embodiment, the dual GLP-1/GIP receptor agonist is LBT-2000 from Longevity Biotech, Inc.

In a further embodiment, the dual GLP-1/GIP receptor agonist is ZPDI-70 from Zealand Pharma A/S.

In an additional embodiment, the dual GLP-1/GIP receptor agonist is a synthetic peptide to agonize GLP-1, GIP and glucagon receptors available from Diabetica Limited.

In a further embodiment, the dual GLP-1/GIP receptor agonist is a small molecule to agonize GLP-1 and GIP receptors from Carmot Therapeutics, Inc.

In some embodiments, the therapeutic methods further comprise administering existing therapies sequentially or simultaneously with the dual GLP-1/GIP receptor agonist described herein.

### BRIEF DESCRIPTION OF FIGURES

Exemplary embodiments are illustrated in the referenced figures. It is intended that the embodiments and figures disclosed herein are to be considered illustrative rather than restrictive.

FIG. 1 depicts, in accordance with an embodiment of the invention, a diagrammatic illustration of the likely role played by brain insulin resistance in AD.

FIGS. 2A-2B depicts, in accordance with an embodiment of the invention, a diagram of the two mechanisms causing brain insulin resistance in AD.

FIG. 3A-FIG. 3C depicts, in accordance with an embodiment of the invention, that brain insulin resistance in AD is closely associated with cognitive decline. FIG. 3A shows linear regression plots of cognitive score on multiple tests of episodic memory. FIG. 3B shows linear regression plots of cognitive score on multiple tests of working memory. FIG. 3C shows linear regression plots of cognitive score on multiple tests of global cognition. The three panels of this figure derive from Talbot, K. et al., Journal of Clinical Investigation 122: 1316-1338, 2012.

FIG. 4A-FIG. 4B depicts, in accordance with an embodiment of the invention, that brain insulin resistance is marked in mild cognitive impairment (MCI) and in AD dementia (FIG. 4a), as well as in the APP/PS1 transgenic mouse model of AD (FIG. 4b)

FIG. 5 depicts, in accordance with an embodiment of the invention, that peripheral administration of an incretin receptor agonist (liraglutide) substantially reduces hippocampal formation insulin resistance in a transgenic mouse model of AD.

FIG. 6 depicts, in accordance with an embodiment of the invention, that ex vivo exposure of hippocampal formation tissue from MCI and AD dementia cases to 100 nM liraglutide for 30 minutes prior to 1 nM insulin stimulation for 30 minutes significantly improves its insulin responsiveness.

FIG. 7 depicts, in accordance with an embodiment of the invention, that a dual GLP-1/GIP receptor agonist is superior to a single GLP-1 receptor agonist (liraglutide) in reducing one cause of brain insulin resistance in AD, namely elevated basal levels of serine phosphorylated insulin receptor substrate-1.

FIG. 8 depicts, in accordance with an embodiment of the invention, that a dual GLP-1/GIP receptor agonist (dual IRA) is superior to single IRAs (the GLP-1 receptor agonist liraglutide or the GIP receptor agonist D-Ala² GIP) in reducing another cause of brain insulin resistance in AD, namely elevated basal levels of SOCS (suppressor of cytokine signaling 3) associated with the insulin receptor.

FIGS. 9A-9B depicts, in accordance with an embodiment of the invention, that a dual incretin receptor agonist (IRA) is superior to single IRAs (the GLP-1 receptor agonist liraglutide or the GIP receptor agonist D-Ala² GIP) in raising insulin responsiveness of the hippocampal formation from amnestic MCI (aMCI) and AD dementia (ADd) cases.

FIG. 10 depicts, in accordance with an embodiment of the invention, that a dual incretin receptor agonist (dual IRA) can cross the blood brain barrier at a significant rate, though less quickly than the single IRA exenatide, a GLP-1 receptor agonist.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed, revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and prolonging a patient's life or life expectancy. Beneficial or desired clinical results include, but are not limited to, alleviation of one or more symptom(s), diminishment of extent of the deficit, stabilized (i.e., not worsening) state of progression, delay or slowing of progression or invasiveness, and amelioration or palliation of symptoms associated with the brain insulin resistance. Treatment also includes a decrease in mortality or an increase in the lifespan of a subject as compared to one not receiving the treatment.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

The term “effective amount” as used herein refers to the amount of the one or more incretin receptor agonists as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering the peptide. Measured or measurable parameters include clinically detectable markers of disease, for example, elevated or depressed levels of a biological marker, as well as parameters related to a clinically accepted scale of symptoms or markers for brain insulin resistance and/or related diseases. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

As used herein, a “subject” means a human or animal. Usually the animal is a vertebrate such as a primate, rodent, domestic animal or game animal. Primates include chimpanzees and cynomologous, spider, and macaque monkeys. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. The term does not denote a particular age or sex. Thus, adult and newborn subjects, as well as fetuses, whether male or female, are intended to be included within the scope of this term.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as brain insulin resistance, mild cognitive impairment (MCI) and/or Alzheimer's disease (AD) dementia. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

“Therapeutic agents” as used herein refers to agents that are used to, for example, treat, inhibit, prevent, mitigate the effects of, reduce the severity of, reduce the likelihood of developing and/or slow the progression of, a disease. Diseases targeted by the therapeutic agents include but are not limited to diseases associated with brain insulin resistance, for example, mild cognitive impairment, Alzheimer's disease dementia, Parkinson's disease, and/or traumatic brain injury.

“Pharmaceutically acceptable carriers” as used herein refer to conventional pharmaceutically acceptable carriers useful in this invention.

“Peptidomimetic” as used herein is a small protein-like chain designed to mimic a protein function. They may be modifications of an existing peptide or newly designed to mimic known peptides. They may be, for example, peptoids and/or β-peptides and/or D-peptides.

“Polynucleotide” as used herein includes but is not limited to DNA, RNA, cDNA (complementary DNA), mRNA (messenger RNA), rRNA (ribosomal RNA), shRNA (small hairpin RNA), snRNA (small nuclear RNA), snoRNA (short nucleolar RNA), miRNA (microRNA), genomic DNA, synthetic DNA, synthetic RNA, and/or tRNA.

### Methods of Use

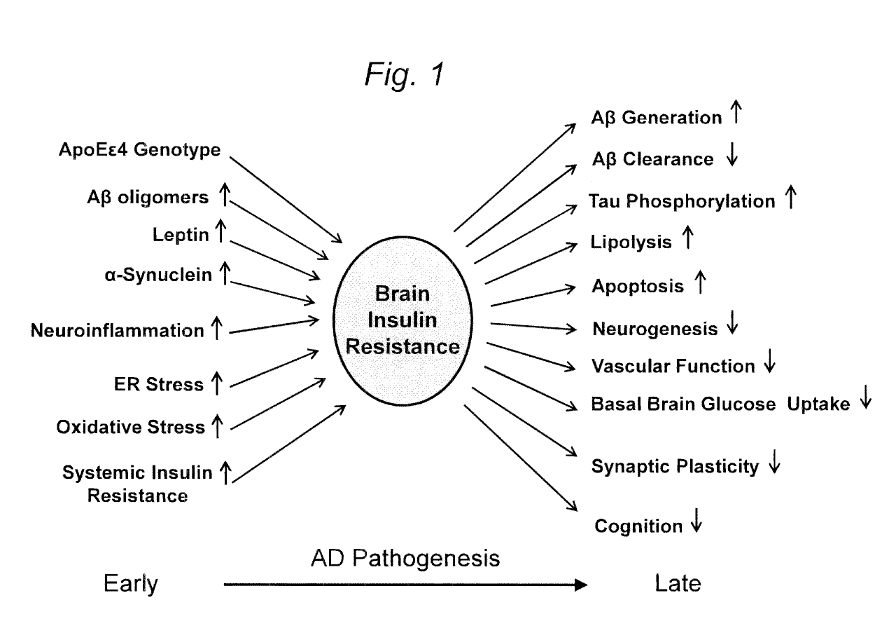

Described herein is the use of a new antidiabetic peptide recently tested in a phase II clinical trial for type 2 diabetes (T2D) as a treatment for brain insulin resistance in general and more specifically as a treatment for mild cognitive impairment (MCI) and the disorder to which it often leads, Alzheimer's disease (AD) dementia. The brain in MCI and AD dementia is commonly and markedly insulin resistant even in the absence of diabetes. This phenomenon can be induced by several early pathogenic factors in AD and can in turn cause or aggravate many of its later pathological features and cognitive deficits (FIG. 1). Brain insulin resistance has consequently emerged as a nodal abnormality in MCI and AD, one which is an efficient target for slowing or even stopping progression of MCI and AD dementia.

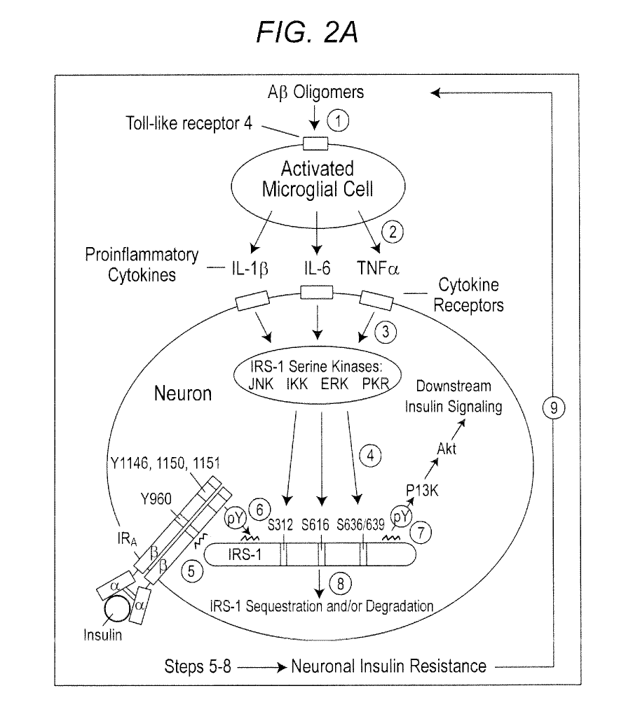

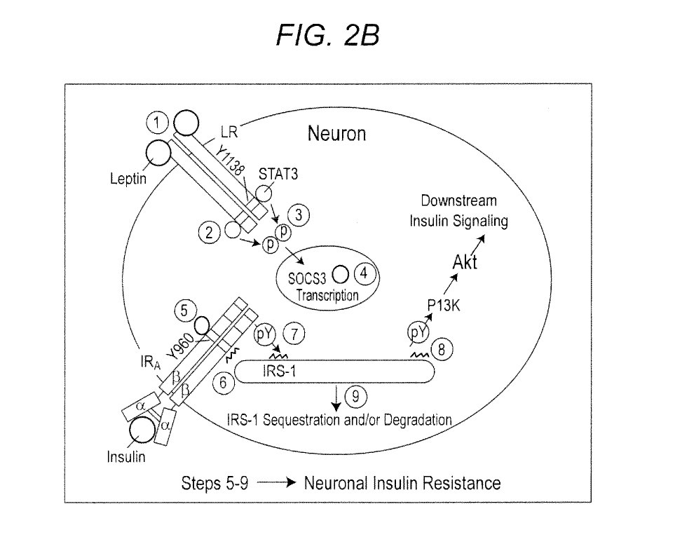

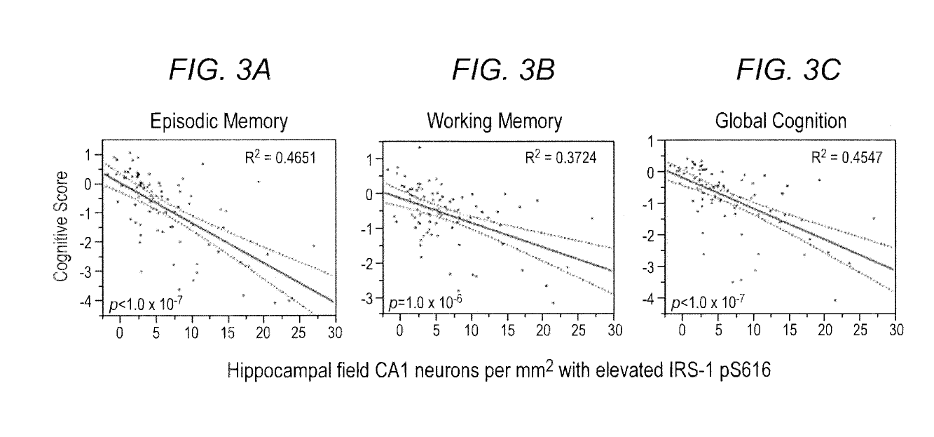

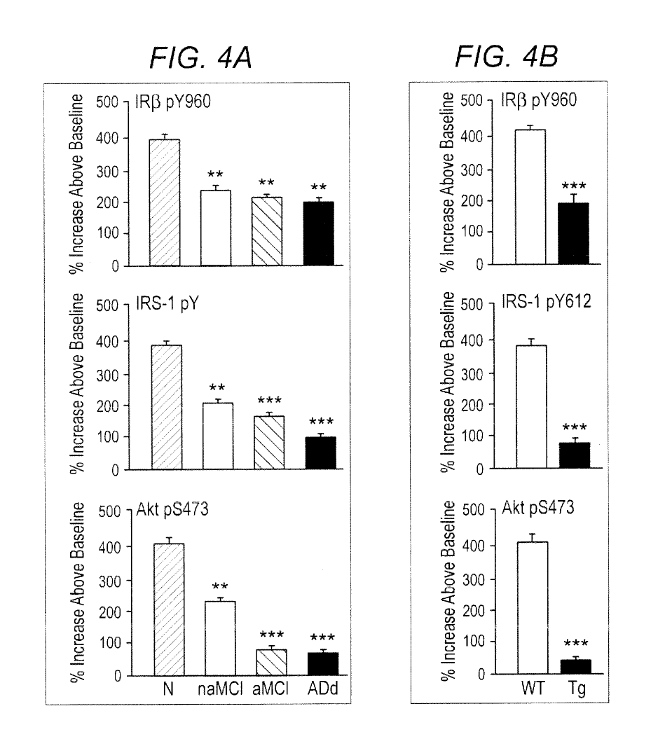

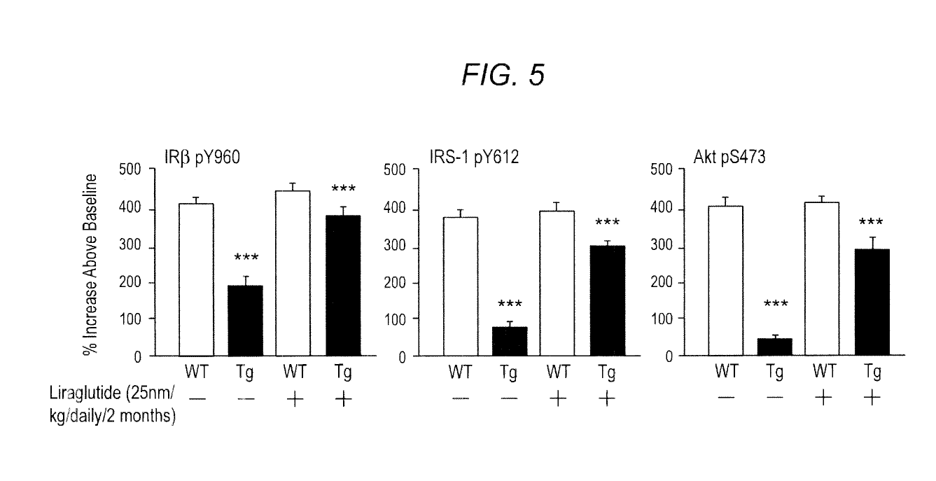

A major cause of brain insulin resistance is serine phosphorylation of insulin receptor substrate-1 (IRS-1 pS), which inhibits insulin signaling. Via one of two neuronal insulin resistance mechanisms shown in FIGS. 2A-2B, amyloid beta (Aβ) oligomers accumulating in the brains of AD cases can drive elevation in IRS-1 pS, several forms of which are biomarkers of brain insulin resistance very closely associated with cognitive decline (FIG. 3). Since neuronal insulin resistance can lead to higher levels of Aβ oligomers, there is a reciprocal relationship between brain insulin resistance and AD pathogenesis (FIG. 1 and FIGS. 2A-2B). This is especially noteworthy given our recent discovery that brain insulin resistance is already advanced in MCI cases (FIG. 4a) and by 7 months in the APP/PS1 mouse model of AD (FIG. 4b). Accordingly, without being bound to a particular theory, the inventors propose that reducing brain insulin resistance in MCI and AD may be an effective treatment of these disease states. The most practical means of reducing brain insulin resistance is with antidiabetics, since type 2 diabetes (T2D) is a risk factor for MCI and AD dementia. Antidiabetics can exert therapeutic effects in AD indirectly and directly. It can do so indirectly by reducing insulin resistance outside the central nervous system (i.e., peripheral or systemic insulin resistance). This peripheral resistance is common in AD dementia and can by itself induce brain insulin resistance. Certain antidiabetics can also directly reduce brain insulin resistance (e.g., FIG. 5-FIG. 7).

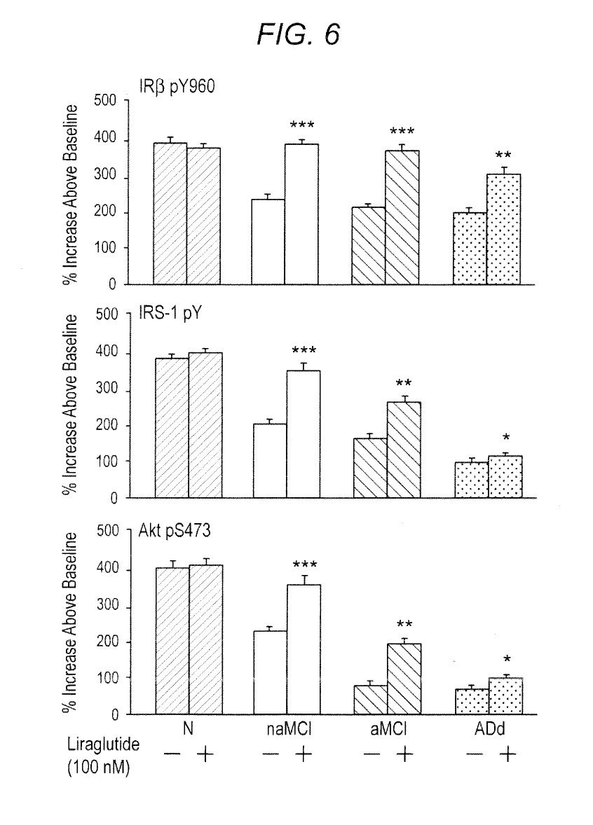

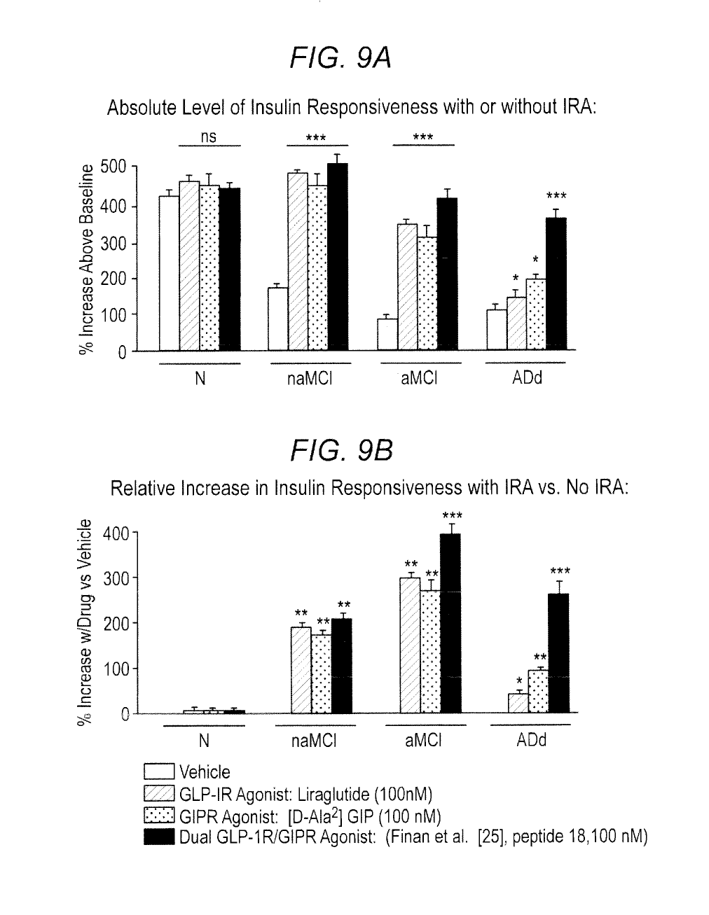

Among the most promising antidiabetics proposed as AD therapeutics are those belonging to the class known as incretin receptor agonists (IRAs). The incretins are peptides so-named because they are intestinal hormones that increase glucose-stimulated secretion of insulin from the pancreas. IRAs are long-acting analogues or mimetics of human incretins, which include gastric inhibitory peptide (GIP, also known as glucose-dependent insulinotropic peptide), glucagon-like peptide 1 (GLP-1), and GLP-2. Single and dual IRAs have been developed that activate the GIP receptor alone (e.g., the single IRAs exenatide and liraglutide) [Tatarkiewicz, K. et al. Diabetes, Obesity and Metabolism 16: 75-85, 2014], the GLP-1 receptor alone (D-Ala² GIP) [Gupta, V. et al. Indian Journal of Endocrinology and Metabolism 17: 413-421, 2013], or both the GIP and GLP-1 receptors equally [Finan, B. et al. Science Translational Medicine 5: 209ra151, 2013]. In addition to reducing peripheral insulin resistance, these are the only drugs of any type known to do all the following: (1) substantially reduce insulin resistance in brains from an AD mouse model (FIG. 5), MCI cases (FIGS. 6 and 9A-9B), and AD dementia cases (FIG. 6-9A-9B), (2) markedly reduce many AD-related brain pathologies, as well as synaptic dysfunction and cognitive deficits in a mouse model of AD, and (3) halt decline in glucose metabolism in AD dementia cases. This last effect has just been reported in a clinical trial on AD cases treated with liraglutide [Gj el, M. et al., Frontiers in Aging Neuroscience 8: 108, 2016]. The IRAs may have similar therapeutic potential in other neurological disorders. The single IRA exenatide has been found to reduce motor symptoms and cognitive deficits in Parkinson's disease [Aviles-Olmos, I. et al., Journal of Clinical Investigation 123: 2730-2736, 2013], where there is now evidence of brain insulin resistance (Talbot, K., in preparation). Single and dual IRAs have also been found to be neuroprotective in animal models of traumatic brain injury [Tamargo et al. Experimental Neurology (in press)].

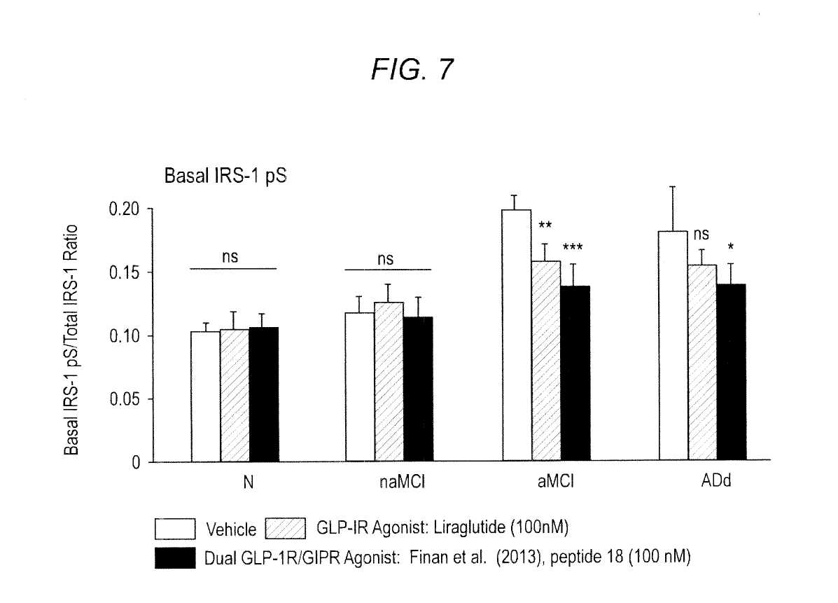

Dual IRAs have unique advantages over single IRAs as AD therapeutics. First, dual GLP-1/GIP receptor agonists are better tolerated than GLP-1 receptor agonists. The one common adverse effect of single IRAs is gastrointestinal upset which appears to be the result of increased gut motility and which is most often experienced as mild to moderate nausea by up to 35% of people during their first 1-4 months even at relatively low doses of the drugs. In contrast, dual IRAs do not increase gut motility and induce mild to moderate nausea only at a high dose, which induces nausea in only 16% of people these drugs. Second, dual GLP-1/GIP receptor agonists are significantly more potent than single IRAs in reducing systemic insulin resistance. Third, in tissue from MCI and AD dementia cases, a dual IRA is superior to single IRAs in brain insulin resistance as seen at the first level of insulin signaling (IRS-1) severely impaired in these cases (FIGS. 7-9A-9B). This is especially striking in AD dementia cases, where only the dual IRA substantially reduces brain insulin resistance substantially.

Accordingly, provided herein are methods for treating brain insulin resistance and diseases associated therewith using dual GLP-1/GIP receptor agonists.

Provided herein is a method for treating, inhibiting, reducing the severity of and/or slowing disease progression in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow disease progression in the subject. In further embodiments, the method includes administering a therapeutically effective amount of the incretin receptor agonist (IRA) or derivatives, pharmaceutical equivalents, peptidomimetics or analogs thereof, so as to treat, inhibit, reduce the severity of and/or slow disease progression in the subject. In one embodiment, the subject has diabetes. In another embodiment, the subject does not have diabetes. In some embodiments, the disease-state is any disease treatable by dual GLP-1/GIP receptor agonist. In one embodiment, the disease-state is brain insulin resistance. In an embodiment, the disease-state is a disease associated with brain insulin resistance. In an embodiment, the disease-state is mild cognitive impairment (MCI). In a further embodiment, the disease-state is Alzheimer's disease (AD) dementia. In an embodiment, the disease-state is Parkinson's disease. In an additional embodiment, the disease-state is traumatic brain injury.

Also provided herein is a method of treating, inhibiting, reducing the severity of and/or slowing progression of brain insulin resistance in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow progression of brain insulin resistance in the subject. In further embodiments, the method includes administering a therapeutically effective amount of an incretin receptor agonist (IRA) or derivatives, pharmaceutical equivalents, peptidomimetics or analogs thereof, so as to treat, inhibit, reduce the severity of and/or slow progression of brain insulin resistance in the subject. In an embodiment, the incretin receptor agonist is dual GLP-1/GIP receptor agonist. In one embodiment, the subject has diabetes. In another embodiment, the subject does not have diabetes.

Further provided herein is a method of treating, inhibiting, reducing the severity of and/or slowing progression of mild cognitive impairment in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow progression of mild cognitive impairment in the subject. In further embodiments, the method includes administering a therapeutically effective amount of an incretin receptor agonist (IRA) or derivatives, pharmaceutical equivalents, peptidomimetics or analogs thereof, so as to treat, inhibit, reduce the severity of and/or slow progression of mild cognitive impairment in the subject. In an embodiment, the incretin receptor agonist is dual GLP-1/GIP receptor agonist. In one embodiment, the subject has diabetes. In another embodiment, the subject does not have diabetes.

Also provided herein is a method of treating, inhibiting, reducing the severity of and/or slowing progression of Alzheimer's disease dementia in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow progression of Alzheimer's disease dementia in the subject. In further embodiments, the method includes administering a therapeutically effective amount of an incretin receptor agonist (IRA) or derivatives, pharmaceutical equivalents, peptidomimetics or analogs thereof, so as to treat, inhibit, reduce the severity of and/or slow progression of Alzheimer's disease dementia in the subject. In an embodiment, the incretin receptor agonist is dual GLP-1/GIP receptor agonist. In one embodiment, the subject has diabetes. In another embodiment, the subject does not have diabetes.

Also provided herein is a method of treating, inhibiting, reducing the severity of and/or slowing progression of Parkinson's disease in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow progression of Parkinson's disease in the subject. In further embodiments, the method includes administering a therapeutically effective amount of an incretin receptor agonist (IRA) or derivatives, pharmaceutical equivalents, peptidomimetics or analogs thereof, so as to treat, inhibit, reduce the severity of and/or slow progression of Parkinson's disease in the subject. In an embodiment, the incretin receptor agonist is dual GLP-1/GIP receptor agonist. In one embodiment, the subject has diabetes. In another embodiment, the subject does not have diabetes.

Also provided herein is a method of treating, inhibiting, reducing the severity of and/or slowing progression of traumatic brain injury (TBI) in a subject in need thereof. In some embodiments, the method comprises providing an incretin receptor agonist (IRA) (for example, dual GLP-1/GIP receptor agonist) or derivatives, pharmaceutical equivalents, peptidomimetics or analogs thereof and administering a therapeutically effective amount of the IRA to the subject so as to treat, inhibit, reduce the severity of and/or slow progression of traumatic brain injury in the subject. In further embodiments, the method includes administering a therapeutically effective amount of an incretin receptor agonist (IRA) or derivatives, pharmaceutical equivalents, peptidomimetics or analogs thereof, so as to treat, inhibit, reduce the severity of and/or slow progression of traumatic brain injury in the subject. In an embodiment, the incretin receptor agonist is dual GLP-1/GIP receptor agonist. In one embodiment, the subject has diabetes. In another embodiment, the subject does not have diabetes.

In some embodiments, the therapeutic methods described herein include diagnosing brain insulin resistance, mild cognitive impairment and/or Alzheimer's disease in the subject and subsequently treating brain insulin resistance, mild cognitive impairment and/or Alzheimer's disease by the methods described herein.

In one embodiment, the dual GLP-1/GIP receptor agonist is an unmodified peptide comprising, consisting of or consisting essentially of the sequence YXEGTFTSDYSIYLDKQAAXEFVNWLLAGGPSSGAPPPSC-NH₂ (SEQ ID NO.: 1), wherein X is aminoisobutyric acid, (for example, peptide 17 in Finan, B. et al. Science Translational Medicine 5: 209ra151, 2013). In some embodiments, the peptide having SEQ ID NO: 1 is acylated or PEGylated.

In another embodiment, the dual GLP-1/GIP receptor agonist is a peptide comprising, consisting of or consisting essentially of the sequence YXEGTFTSDYSIYLDKQAAXEFVNWLLAGGPSSGAPPPSK (SEQ ID NO.: 2), (for example, peptide 18 in Finan, B. et al. Science Translational Medicine 5: 209ra151, 2013), wherein X is aminoisobutyric acid. In some embodiments, the peptides having the sequence of SEQ ID NO: 2 have been acylated or PEGylated. In one such embodiment, the peptide having SEQ ID NO: 2 is acylated at Lys-40 ((K⁴⁰-C₁₆ acyl)-NH₂), (for example, compound 19 in Finan, B. et al. Science Translational Medicine 5: 209ra151, 2013).

In a further embodiment, the peptide having SEQ ID NO:2 is PEGylated at Cys-24 (for example, compound 21 in Finan, B. et al. Science Translational Medicine 5: 209ra151, 2013).

In another embodiment, the dual GLP-1/GIP receptor agonist is LBT-2000 from Longevity Biotech, Inc. In a further embodiment, the dual GLP-1/GIP receptor agonist is ZPDI-70 from Zealand Pharma A/S. In an additional embodiment, the dual GLP-1/GIP receptor agonists are synthetic peptides to agonize GLP-1, GIP and glucagon receptors available from Diabetica Limited. In a further embodiment, the dual GLP-1/GIP receptor agonists are small molecules to agonize GLP-1 and GIP Receptors from Carmot Therapeutics, Inc.

In some embodiments of the therapeutic methods described herein, the dual receptor agonists described herein are used alone or in combinations. For example, peptides having the sequence of SEQ ID NO: 1, peptides having the sequence of SEQ ID NO: 2, peptides having the sequence of SEQ ID NO: 3 may be used alone or in combinations thereof. In further exemplary embodiments, peptides having the sequence of SEQ ID NO: 1, peptides having the sequence of SEQ ID NO: 2, peptides having the sequence of SEQ ID NO: 3, LBT-2000, ZPD1-70, the synthetic peptides from Diabetica Limited and small molecules from Carmot Therapeutics, Inc. may each be used alone or in combinations thereof for the therapeutic methods described herein.

In some embodiments, the therapeutic methods described herein further comprise administering additional therapies including but not limited to any one or more of cholinesterase inhibitors, tacrine (Cognex), NMDA receptor antagonist or a combination thereof. In exemplary embodiments, the cholinesterase inhibitor is donepezil (Aricept), galantamine (Razadyne) or rivastigmine (Exelon). In exemplary embodiments, the NMDA receptor antagonist is memantine (Namenda). In some embodiments, the additional therapies are administered simultaneously with dual GLP-1/GIP receptor agonists described herein. In some embodiments, the additional therapies are administered sequentially with dual GLP-1/GIP receptor agonists described herein. In some embodiments, the dual GLP-1/GIP receptor agonists described herein are administered first for a period of time and the additional therapies are administered thereafter. In some embodiments, the additional therapies are administered after treatment with dual GLP-1/GIP receptor agonists described herein. The combinations and orders of treatment comprising the additional therapies and the dual GLP-1/GIP receptor agonists described herein will be apparent to a person of skill in the art.

In some embodiments of the therapeutic methods described herein, the dual GLP-1/GIP receptor agonists described herein are administered intravenously, intramuscularly, intraperitonealy or subcutaneously, orally or via inhalation. In one embodiment, dual GLP-1/GIP receptor agonists described herein are administered intraperitonealy. In another embodiment, dual GLP-1/GIP receptor agonists described herein are administered subcutaneously. In various embodiments, the composition comprising the dual GLP-1/GIP receptor agonist is administrated to the subject before, during, or after the subject develops the disease-state (for example, brain insulin resistance, mild cognitive impairment, Alzheimer's disease dementia, Parkinson's disease, and/or traumatic brain injury).

In exemplary embodiments, the composition comprising the dual GLP-1/GIP receptor agonist is administrated to the subject 1-3 times per day or 1-7 times per week. In some embodiments, he dual GLP-1/GIP receptor agonist is administrated to the subject once a day (QD) after first testing tolerability of the drug. In some embodiments, the composition comprising the dual GLP-1/GIP receptor agonist is administrated to the subject for 1-5 days, 1-5 weeks, 1-5 months, 3-6 months, 4-10 months, or 1-5 years. In some embodiments, the composition comprising the dual GLP-1/GIP receptor agonist is administrated to the subject chronically for at least 3-5 months, 4-6 months, 5-7 months, 6-8 months, 7-9 months, 8-10 months, 9-11 months, 10-12 months or combinations thereof. In one embodiment, the composition comprising the dual GLP-1/GIP receptor agonist is administrated to the subject chronically for about 6 months. In an embodiment, the composition comprising the dual GLP-1/GIP receptor agonist is administrated to the subject chronically for at least 6 months. In some embodiments, the composition comprising the dual GLP-1/GIP receptor agonist is administrated to the subject for the life of the subject.

In various embodiments, the effective amount of the incretin receptor agonist (for example, dual GLP-1/GIP receptor agonist) is any one or more of about 0.01 to 0.05 μg/kg/day, 0.05-0.1 μg/kg/day, 0.1 to 0.5 μg/kg/day, 0.5 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. In an exemplary embodiment, the effective amount of the incretin receptor agonist (for example, dual GLP-1/GIP receptor agonist) is about 10 to 110 mg/kg body weight per day and the agonist is administered for about 3-8 weeks. Typical dosages of an effective amount of incretin receptor agonist (for example, dual GIP receptor and GLP-1 receptor agonist) can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, such as biopsied malignant tumors, or the responses observed in the appropriate animal models. In various embodiments, the compositions of the invention comprising the incretin receptor agonist (for example, a dual GIP receptor and a GLP-1 receptor agonist) may be administered once a day (SID/QD), twice a day (BID), three times a day (TID), four times a day (QID), or more, so as to administer an effective amount of the incretin receptor agonist (for example, dual GIP receptor and GLP-1 receptor agonist) to the subject, where the effective amount is any one or more of the doses described herein.

In various embodiments, the subject is selected from the group consisting of human, non-human primate, monkey, ape, dog, cat, cow, horse, rabbit, mouse and rat.

### Diagnostic Methods

Also provided herein are methods for determining the presence of brain insulin resistance in a subject in need thereof. The methods include obtaining a sample (for example, brain biopsy, cerebrospinal fluid [CSF], neuronally-derived exosomes in blood) from the subject; assaying the sample for an increase in level of serine phosphorylated insulin receptor substrate-1(IRS-1 pS) relative to reference value, wherein the IRS-1 is phosphorylated at one or more serine residues, and determining that the subject has an increased likelihood of brain insulin resistance if there is an increase in the amount of the IRS-1pS relative to reference value.

Also provided herein is a method for determining the likelihood of mild cognitive impairment in a subject in need thereof. The methods include determining the presence of brain insulin resistance in the subject by the methods described herein and determining that the subject has an increased likelihood of mild cognitive impairment if the subject has an increased likelihood of brain insulin resistance, as measured by the increase in levels of IRS-1 pS.

Further provided herein is a method for determining the likelihood of Alzheimer's disease dementia in a subject in need thereof. The methods include determining the presence of brain insulin resistance in the subject by the methods described herein and determining that the subject has an increased likelihood of Alzheimer's disease dementia if the subject has an increased likelihood of brain insulin resistance, as measured by the increase in levels of IRS-1 pS.

Also provided herein are methods for determining the efficacy of treatment for brain insulin resistance in a subject in need thereof. The methods include obtaining a sample for the subject; assaying the sample to determine the levels of IRS-1 pS; and determining that the treatment is effective if the level of the IRS-1 pS is decreased relative to the reference value.

In various embodiments, assaying comprises using is immunoassays. Exemplary embodiments of immunoassays include but are not limited to any one or more of ELISA, RIA, Western blotting, Southern blotting, or combinations thereof. In one embodiment, an ELISA is used to measure IRS-1 pS species.

In some embodiments of the assays/diagnostic methods described herein, the sample is any one or more of brain, CSF, neuronally-derived exosomes in blood, plasma, blood, urine, tissue or combinations thereof. In some embodiments, the sample (for example, the blood or plasma sample) comprises tissues derived from the brain (for example, brain-derived exosomes). These samples may also be tested for biomarkers of brain insulin resistance, mild cognitive impairment, Alzheimer's disease dementia, Parkinson's disease, and/or traumatic brain injury. In some embodiments, the sample is obtained before, during or after treatment for brain insulin resistance.

In one embodiment of the methods described herein, the subject is human.

Any suitable immunoassay method may be utilized, including those which are commercially available, to determine the level IRS-1 pS specific antibodies measured according to the embodiments of the instant invention. Extensive discussion of the known immunoassay techniques is not required here since these are known to those of skill in the art. Typical suitable immunoassay techniques include sandwich enzyme-linked immunoassays (ELISA), radioimmunoassays (MA), competitive binding assays, homogeneous assays, heterogeneous assays, etc. Various known immunoassay methods are reviewed, e.g., in Methods in Enzymology, 70, pp. 30-70 and 166-198 (1980).

In the assays of the invention, “sandwich-type” assay formats can be used. Some examples of such sandwich-type assays are described in by U.S. Pat. No. 4,168,146 to Grubb, et al. and U.S. Pat. No. 4,366,241 to Tom, et al. An alternative technique is the “competitive-type” assay. In a competitive assay, the labeled probe is generally conjugated with a molecule that is identical to, or an analog of, the analyte. Thus, the labeled probe competes with the analyte of interest for the available receptive material. Competitive assays are typically used for detection of analytes such as haptens, each hapten being monovalent and capable of binding only one antibody molecule. Examples of competitive immunoassay devices are described in U.S. Pat. No. 4,235,601 to Deutsch, et al., U.S. Pat. No. 4,442,204 to Liotta, and U.S. Pat. No. 5,208,535 to Buechler, et al.

The antibodies can be labeled. In some embodiments, the detection antibody is labeled by covalently linking to an enzyme, label with a fluorescent compound or metal, label with a chemiluminescent compound. For example, the detection antibody can be labeled with catalase and the conversion uses a colorimetric substrate composition comprises potassium iodide, hydrogen peroxide and sodium thiosulphate; the enzyme can be alcohol dehydrogenase and the conversion uses a colorimetric substrate composition comprises alcohol, a pH indicator and a pH buffer, wherein the pH indicator is neutral red and the pH buffer is glycine-sodium hydroxide; the enzyme can also be hypoxanthine oxidase and the conversion uses a colorimetric substrate composition comprises xanthine, a tetrazolium salt and 4,5-dihydroxy-1,3-benzene disulphonic acid. In one embodiment, the detection antibody is labeled by covalently linking to an enzyme, label with a fluorescent compound or metal, or label with a chemiluminescent compound.

Direct and indirect labels can be used in immunoassays. A direct label can be defined as an entity, which in its natural state, is visible either to the naked eye or with the aid of an optical filter and/or applied stimulation, e.g., ultraviolet light, to promote fluorescence. Examples of colored labels which can be used include metallic sol particles, gold sol particles, dye sol particles, dyed latex particles or dyes encapsulated in liposomes. Other direct labels include radionuclides and fluorescent or luminescent moieties. Indirect labels such as enzymes can also be used according to the invention. Various enzymes are known for use as labels such as, for example, alkaline phosphatase, horseradish peroxidase, lysozyme, glucose-6-phosphate dehydrogenase, lactate dehydrogenase and urease. For a detailed discussion of enzymes in immunoassays see Engvall, Enzyme Immunoassay ELISA and EMIT, Methods of Enzymology, 70, 419-439 (1980).

The antibody can be attached to a surface. Examples of useful surfaces on which the antibody can be attached for the purposes of detecting the desired antigen include nitrocellulose, PVDF, polystyrene, and nylon. The surface or support may also be a porous support (see, e.g., U.S. Pat. No. 7,939,342). The assays can be carried out in various assay device formats including those described in U.S. Pat. Nos. 4,906,439; 5,051,237 and 5,147,609 to PB Diagnostic Systems, Inc.

### Reference Values

In various embodiments of the assays described herein, the reference value is based on the levels of IRS-1 pS in the sample. In one embodiment, the reference value is the mean or median level of IRS-1 pS in a population of subjects that do not have brain insulin resistance. In another embodiment, the reference value is the mean or median level of IRS-1 pS in a sample obtained from the subject at a different time point (for example, prior to starting treatment). In a further embodiment, the reference value is the mean or median level of IRS-1 pS in a population of subjects that have been successfully treated for brain insulin resistance.

In various embodiments of the diagnostic methods described herein, the levels of IRS-1 pS in the subject compared to the reference values is increased by at least or about 10%, 20%, 30%, 40%, 50%, 60%, 70%, 80%, 90% or 100%. In various embodiments, the levels of IRS-1 pS in the subject compared to the reference values is increased by at least or about 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold or 100-fold.

In various embodiments of the methods for determining efficacy of treatment, the levels of IRS-1 pS in the subject compared to the reference values is decreased by at least or about 10%, 20%, 30%, 40%, 50%, 60%, 70%, 80%, 90% or 100%. In various embodiments, the levels of IRS-1 pS in the subject compared to the reference values is decreased by at least or about 1-fold, 2-fold, 3-fold, 4-fold, 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold or 100-fold.

### Dual GLP-1/GIP Receptor Agonists

In some embodiments, the dual GLP-1/GIP receptor agonists described herein or combinations thereof, or analogs, pharmaceutical equivalents and/or peptidomimetics thereof are modified peptides. “Modified peptide” may include the incorporation of lactam-bridge, head-to-tail cyclization, non-natural amino acids into the peptides of the invention, including synthetic non-native amino acids, substituted amino acids, or one or more D-amino acids into the peptides (or other components of the composition, with exception for protease recognition sequences) is desirable in certain situations. D-amino acid-containing peptides exhibit increased stability in vitro or in vivo compared to L-amino acid-containing forms. Thus, the construction of peptides incorporating D-amino acids can be particularly useful when greater in vivo or intracellular stability is desired or required. More specifically, D-peptides are resistant to endogenous peptidases and proteases, thereby providing better oral trans-epithelial and transdermal delivery of linked drugs and conjugates, improved bioavailability of membrane-permanent complexes (see below for further discussion), and prolonged intravascular and interstitial lifetimes when such properties are desirable. The use of D-isomer peptides can also enhance transdermal and oral trans-epithelial delivery of linked drugs and other cargo molecules. Additionally, D-peptides cannot be processed efficiently for major histocompatibility complex class II-restricted presentation to T helper cells, and are therefore less likely to induce humoral immune responses in the whole organism. Peptide conjugates can therefore be constructed using, for example, D-isomer forms of cell penetrating peptide sequences, L-isomer forms of cleavage sites, and D-isomer forms of therapeutic peptides. Therefore, in some embodiments the peptides as disclosed comprise L and D amino acids, wherein no more than 1, 2, 3, 4, 5, 6, 7, 8, 9, or 10 D-amino acids are included. In certain aspects, the peptides comprise more than 10 D-amino acids, and in certain aspects all the amino acids of the peptides are D-amino acids.

In some embodiments, the dual GLP-1/GIP receptor agonists described herein or combinations thereof, or analogs, pharmaceutical equivalents and/or peptidomimetics thereof are retro-inverso peptides. A “retro-inverso peptide” refers to a peptide with a reversal of the direction of the peptide bond on at least one position, i.e., a reversal of the amino- and carboxy-termini with respect to the side chain of the amino acid. Thus, a retro-inverso analogue has reversed termini and reversed direction of peptide bonds while approximately maintaining the topology of the side chains as in the native peptide sequence. The retro-inverso peptide can contain L-amino acids or D-amino acids, or a mixture of L-amino acids and D-amino acids, up to all of the amino acids being the D-isomer. Partial retro-inverso peptide analogues are polypeptides in which only part of the sequence is reversed and replaced with enantiomeric amino acid residues. Since the retro-inverted portion of such an analogue has reversed amino and carboxyl termini, the amino acid residues flanking the retro-inverted portion are replaced by side-chain-analogous α-substituted geminal-diaminomethanes and malonates, respectively. Retro-inverso forms of cell penetrating peptides have been found to work as efficiently in translocating across a membrane as the natural forms. Synthesis of retro-inverso peptide analogues are described in Bonelli, F. et al., Int J Pept Protein Res. 24(6):553-6 (1984); Verdini, A and Viscomi, G. C, J. Chem. Soc. Perkin Trans. 1:697-701 (1985); and U.S. Pat. No. 6,261,569, which are incorporated herein in their entirety by reference. Processes for the solid-phase synthesis of partial retro-inverso peptide analogues have been described (EP 97994-B) which is also incorporated herein in its entirety by reference.

Other variants of the dual GLP-1/GIP receptor agonists described herein can comprise conservatively substituted sequences, meaning that one or more amino acid residues of an original peptide are replaced by different residues, and that the conservatively substituted peptide retains a desired biological activity, i.e., the ability to treat brain insulin resistance, mild cognitive impairment and/or Alzheimer's disease dementia that is essentially equivalent to that of the original peptide. Examples of conservative substitutions include substitution of amino acids that do not alter the secondary and/or tertiary structure of the dual GLP-1/GIP receptor agonists described herein or combinations thereof, or analogs, pharmaceutical equivalents and/or peptidomimetics thereof, substitutions that do not change the overall or local hydrophobic character, substitutions that do not change the overall or local charge, substitutions by residues of equivalent side chain size, or substitutions by side chains with similar reactive groups.

Other examples involve substitution of amino acids that have not been evolutionarily conserved in the parent sequence across species. Advantageously, in some embodiments, these conserved amino acids and structures are not altered when generating conservatively substituted sequences.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar side chain volume are well known. Isolated peptides comprising conservative amino acid substitutions can be tested in any one of the assays described herein to confirm that a desired activity (e.g. reducing brain insulin resistance and treating mild cognitive impairment, Alzheimer's disease dementia, Parkinson's disease and/or traumatic brain injury) is retained as determined by the assays apparent to a person of skill in the art.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

Particularly preferred conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu. In general, conservative substitutions encompass residue exchanges with those of similar physicochemical properties (i.e. substitution of a hydrophobic residue for another hydrophobic amino acid).

Any cysteine residue not involved in maintaining the proper conformation of the isolated peptide as described herein can also be substituted, generally with serine, to improve the oxidative stability of the molecule and prevent aberrant crosslinking. Conversely, cysteine bond(s) can be added to the isolated peptide as described herein to improve its stability or facilitate multimerization.

As used herein, a “functional fragment” is a fragment or segment of a peptide comprising at least 3, at least 4 or at least 5 amino acids and which can treat and/or reduce brain insulin resistance, mild cognitive impairment and/or Alzheimer's disease dementia. A functional fragment can comprise conservative substitutions of the sequences disclosed herein so long as they preserve the function of treating and/or reducing brain insulin resistance, mild cognitive impairment, Alzheimer's disease dementia, Parkinson's disease, and/or traumatic brain injury.

To enhance stability, bioavailability, and/or delivery of the peptides (the dual GLP-1/GIP receptor agonists) into the cells, the peptides can be modified. For example, in some embodiments, the dual GLP-1/GIP receptor agonists as described herein can comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. The dual GLP-1/GIP receptor agonists as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof. In some embodiments, the dual GLP-1/GIP receptor agonists described herein or combinations thereof, or analogs, pharmaceutical equivalents and/or peptidomimetics thereof, are conjugated with agents that increase retention in the body. Examples of agents that increase retention include but are not limited to cellulose, fatty acids, polyethylene glycol (PEG) or combinations thereof.

In some embodiments, the dual GLP-1/GIP receptor agonists as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). In some embodiments, the dual GLP-1/GIP receptor agonists as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, the dual GLP-1/GIP receptor agonists can be modified, e.g. a moiety can be added to one or more of the amino acids comprising the agonists. In some embodiments, the dual GLP-1/GIP receptor agonists as described herein can comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. In some embodiments, the dual GLP-1/GIP receptor agonists as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties.

The dual GLP-1/GIP receptor agonists as described herein can be coupled and or connected to a second functional molecule, peptide and/or polypeptide. In some embodiments, the one or more agonists are coupled to a targeting molecule. In some embodiments, the one or more agonists are coupled to a targeting molecule by expressing the peptide and the targeting molecule as a fusion protein, optionally with a peptide linker sequence interposed between them. As used herein a “targeting molecule” can be any molecule, e.g. a peptide, antibody or fragment thereof, antigen, targeted liposome, or a small molecule that can bind to or be bound by a specific cell or tissue type. By way of non-limiting example, if it is desired to target cells causing brain insulin resistance, dual GLP-1/GIP receptor agonists could be coupled to an antibody or fragment thereof which is specific for the target cell type or region. Methods of synthesizing or producing a fusion protein are well known to those of ordinary skill in the art. The term “fusion protein” as used herein refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. Fusion proteins can include an epitope tag or a half-life extender. Epitope tags include biotin, FLAG tag, c-myc, hemaglutinin, His6, digoxigenin, FITC, Cy3, Cy5, green fluorescent protein, V5 epitope tags, GST, β-galactosidase, AU1, AU5, and avidin. Half-life extenders include Fc domain and serum albumin.

In some embodiments, the dual GLP-1/GIP receptor agonists as described herein can be a pharmaceutically acceptable prodrug. As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenytoin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), which are incorporated by reference herein in their entireties.

In some embodiments, the dual GLP-1/GIP receptor agonists as described herein can be a pharmaceutically acceptable solvate. The term “solvate” refers to an isolated peptide as described herein in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions.

In some embodiments, the dual GLP-1/GIP receptor agonists as described herein can be in a non-crystalline, i.e. amorphous solid form.

### Pharmaceutical Compositions

Provided herein are pharmaceutical compositions comprising, consisting of or consisting essentially of, a dual GLP-1/GIP receptor agonist and an acceptable carrier/excipient.

In one embodiment, the dual GLP-1/GIP receptor agonist is an unmodified peptide comprising, consisting of or consisting essentially of the sequence YXEGTFTSDYSIYLDKQAAXEFVNWLLAGGPSSGAPPPSC-NH₂ (SEQ ID NO.: 1), wherein X is aminoisobutyric acid, (for example, peptide 17 in Finan, B. et al. Science Translational Medicine 5: 209ra151, 2013). In some embodiments, the peptide having SEQ ID NO: 1 is acylated or PEGylated.

In another embodiment, the dual GLP-1/GIP receptor agonist is a peptide comprising, consisting of or consisting essentially of the sequence YXEGTFTSDYSIYLDKQAAXEFVNWLLAGGPSSGAPPPSK (SEQ ID NO.: 2), (for example, peptide 18 in Finan, B. et al. Science Translational Medicine 5: 209ra151, 2013), wherein X is aminoisobutyric acid. In some embodiments, the peptides having the sequence of SEQ ID NO: 2 have been acylated or PEGylated. In one such embodiment, the peptide having SEQ ID NO: 2 is acylated at Lys-40 ((K⁴⁰-C₁₆ acyl)-NH₂), (for example, compound 19 in Finan, B. et al. Science Translational Medicine 5: 209ra151, 2013).

In some embodiments, the incretin receptor agonist is dual GLP-1 receptor and GIP receptor (GLP-1/GIP) receptor agonist. In one embodiment, the dual GLP-1/GIP receptor agonist is a peptide comprising, consisting of or consisting essentially of the sequence YXEGTFTSDYSIYLDKQAAXEFVNWLLAGGPSSGAPPPSK^{b}—NH₂, as described as peptide 19 in Finan, B. et al. [Science Translational Medicine 5: 209ra151, 2013], wherein X is aminoisobutyric acid and K^{b} is Lys-C₁₆ acyl.

In a further embodiment, the peptide having SEQ ID NO:2 is PEGylated at Cys-24 (for example, compound 21 in Finan, B. et al. Science Translational Medicine 5: 209ra151, 2013).

In another embodiment, the dual GLP-1/GIP receptor agonist is LBT-2000 from Longevity Biotech, Inc. In a further embodiment, the dual GLP-1/GIP receptor agonist is ZPDI-70 from Zealand Pharma A/S. In an additional embodiment, the dual GLP-1/GIP receptor agonist is a synthetic peptide to agonize GLP-1, GIP and glucagon receptors available from Diabetica Limited. In a further embodiment, the dual GLP-1/GIP receptor agonist is a small molecule to agonize GLP-1 and GIP Receptors from Carmot Therapeutics, Inc.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of the dual GLP-1/GIP receptor agonist other than directly into a target site, tissue, or organ, such that it enters the subject's circulatory system and, thus, is subject to metabolism and other like processes.

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

The therapeutic may be administered to the patient in a single dose or in multiple doses. When multiple doses are administered, the doses may be separated from one another by, for example, one hour, three hours, six hours, eight hours, one day, two days, one week, two weeks, or one month. For example, the therapeutic may be administered for, e.g., 2, 3, 4, 5, 6, 7, 8, 10, 15, 20, or more weeks. In various embodiments, the composition is administrated to the subject 1-3 times per day or 1-7 times per week. In various embodiments, the composition is administrated to the subject for 1-5 days, 1-5 weeks, 1-5 months, or 1-5 years. It is to be understood that, for any particular subject, specific dosage regimes should be adjusted over time according to the individual need and the professional judgment of the person administering or supervising the administration of the compositions. For example, the dosage of the therapeutic can be increased if the lower dose does not provide sufficient therapeutic activity. While the attending physician ultimately will decide the appropriate amount and dosage regimen, therapeutically effective amounts of the one or more peptides as disclosed herein or a mutant, variant, analog or derivative thereof may be provided at a dose of 0.0001, 0.01, 0.01 0.1, 1, 5, 10, 25, 50, 100, 500, or 1,000 mg/kg or μg/kg. Effective doses may be extrapolated from dose-response curves derived from in vitro or animal model test bioassays or systems.

An effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent to function as the dual GLP-1/GIP receptor agonist, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” or “pharmaceutically acceptable excipient” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, the dual GLP-1/GIP receptor agonist. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) pH buffered solutions; (19) polyesters, polycarbonates and/or polyanhydrides; (20) bulking agents, such as polypeptides and amino acids (21) serum components, such as serum albumin, HDL and LDL; (22) C2-C12 alchols, such as ethanol; and (23) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein.

The dual GLP-1/GIP receptor agonist described herein can be specially formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) topical application, for example, as a cream, ointment, or a controlled-release patch or spray applied to the skin; (3) intravaginally or intrarectally, for example, as a pessary, cream or foam; (4) ocularly; (5) transdermally; (6) transmucosally; or (7) nasally. Additionally, the dual GLP-1/GIP receptor agonist described herein can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 3,270,960.

Further embodiments of the formulations and modes of administration of the dual GLP-1/GIP receptor agonist that can be used in the methods described herein are illustrated below.

Parenteral Dosage Forms. Parenteral dosage forms of the dual GLP-1/GIP receptor agonist can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions.

Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Aerosol formulations. The dual GLP-1/GIP receptor agonist can be packaged in a pressurized aerosol container together with suitable propellants, for example, hydrocarbon propellants like propane, butane, or isobutane with conventional adjuvants. The dual GLP-1/GIP receptor agonist can also be administered in a non-pressurized form such as in a nebulizer or atomizer. The dual GLP-1/GIP receptor agonist can also be administered directly to the airways in the form of a dry powder, for example, by use of an inhaler.

Suitable powder compositions include, by way of illustration, powdered preparations of the dual GLP-1/GIP receptor agonist thoroughly intermixed with lactose, or other inert powders acceptable for intra-bronchial administration. The powder compositions can be administered via an aerosol dispenser or encased in a breakable capsule which can be inserted by the subject into a device that punctures the capsule and blows the powder out in a steady stream suitable for inhalation. The compositions can include propellants, surfactants, and co-solvents and can be filled into conventional aerosol containers that are closed by a suitable metering valve.

Aerosols for the delivery to the respiratory tract are known in the art. See for example, Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic an diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), contents of all of which are herein incorporated by reference in their entirety.

The formulations of the dual GLP-1/GIP receptor agonist described herein further encompass anhydrous pharmaceutical compositions and dosage forms comprising the disclosed compounds as active ingredients, since water can facilitate the degradation of some compounds. For example, the addition of water (e.g., 5%) is widely accepted in the pharmaceutical arts as a means of simulating long-term storage in order to determine characteristics such as shelf life or the stability of formulations over time. See, e.g., Jens T. Carstensen, Drug Stability: Principles & Practice, 379-80 (2nd ed., Marcel Dekker, NY, N.Y.: 1995). Anhydrous pharmaceutical compositions and dosage forms of the disclosure can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. Anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials) with or without desiccants, blister packs, and strip packs.

Controlled and Delayed Release Dosage Forms. In some embodiments of the methods described herein, the dual GLP-1/GIP receptor agonist can be administered to a subject by controlled- or delayed-release means. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. (Kim, Cherng-ju, Controlled Release Dosage Form Design, 2 (Technomic Publishing, Lancaster, Pa.: 2000)). Controlled-release formulations can be used to control a compound's onset of action, duration of action, plasma levels within the therapeutic window, and peak blood levels. In particular, controlled- or extended-release dosage forms or formulations can be used to ensure that the maximum effectiveness of a compound of formula (I) is achieved while minimizing potential adverse effects and safety concerns, which can occur both from under-dosing a drug (i.e., going below the minimum therapeutic levels) as well as exceeding the toxicity level for the drug.

A variety of known controlled- or extended-release dosage forms, formulations, and devices can be adapted for use with the dual GLP-1/GIP receptor agonist described herein. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1, each of which is incorporated herein by reference in their entireties. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), multilayer coatings, microparticles, liposomes, or microspheres or a combination thereof to provide the desired release profile in varying proportions. Additionally, ion exchange materials can be used to prepare immobilized, adsorbed salt forms of the disclosed compounds and thus effect controlled delivery of the drug. Examples of specific anion exchangers include, but are not limited to, Duolite® A568 and Duolite® AP143 (Rohm&Haas, Spring House, Pa. USA).

In some embodiments, the dual GLP-1/GIP receptor agonist for use in the methods described herein is administered to a subject by sustained release or in pulses. Pulse therapy is not a form of discontinuous administration of the same amount of a composition over time, but comprises administration of the same dose of the composition at a reduced frequency or administration of reduced doses. Sustained release or pulse administrations are particularly preferred when the disorder occurs continuously in the subject, for example where the subject has continuous or chronic symptoms of a viral infection. Each pulse dose can be reduced and the total amount of the dual GLP-1/GIP receptor agonist administered over the course of treatment to the patient is minimized.

The interval between pulses, when necessary, can be determined by one of ordinary skill in the art. Often, the interval between pulses can be calculated by administering another dose of the composition when the composition or the active component of the composition is no longer detectable in the subject prior to delivery of the next pulse. Intervals can also be calculated from the in vivo half-life of the composition. Intervals can be calculated as greater than the in vivo half-life, or 2, 3, 4, 5 and even 10 times greater the composition half-life. Various methods and apparatus for pulsing compositions by infusion or other forms of delivery to the patient are disclosed in U.S. Pat. Nos. 4,747,825; 4,723,958; 4,948,592; 4,965,251 and 5,403,590.

### Kits

The invention provides a kit for treating brain insulin resistance, inhibiting brain insulin resistance, reducing brain insulin resistance or slowing progression of brain insulin resistance in a subject in need thereof. The kit comprises a composition comprising a dual GLP-1/GIP receptor agonist and instructions for use of the composition for treating, inhibiting, reducing and/or slowing progression of, brain insulin resistance in subjects in need thereof.

The invention also provides a kit for treating AD dementia, inhibiting AD dementia, reducing AD dementia or slowing progression of AD dementia in a subject in need thereof. The kit comprises a composition comprising a dual GLP-1/GIP receptor agonist and instructions for use of the composition for treating, inhibiting, reducing and/or slowing progression of, AD dementia in subjects in need thereof.

The invention further provides a kit for treating MCI, inhibiting MCI, reducing MCI or slowing progression of MCI in a subject in need thereof. The kit comprises a composition comprising a dual GLP-1/GIP receptor agonist and instructions for use of the composition for treating, inhibiting, reducing and/or slowing progression of, MCI in subjects in need thereof.

In one embodiments, the dual GLP-1/GIP receptor agonist is a peptide comprising, consisting of or consisting essentially of the sequence YXEGTFTSDYSIYLDKQAAXEFVNWLLAGGPSSGAPPPSK, wherein X is aminoisobutyric acid, as described as peptide 18 in Finan, B. et al. Science Translational Medicine 5: 209ra151, 2013, herein incorporated by reference. In some embodiments, peptide 18 is acylated or PEGylated. In another embodiment, the dual GLP-1/GIP receptor agonist is LBT-2000 from Longevity Biotech, Inc. In a further embodiment, the dual GLP-1/GIP receptor agonist is ZPDI-70 from Zealand Pharma A/S. In an additional embodiment, the dual GLP-1/GIP receptor agonist is a synthetic peptide to agonize GLP-1, GIP and glucagon receptors available from Diabetica Limited. In a further embodiment, the dual GLP-1/GIP receptor agonist is a small molecule to agonize GLP-1 and GIP Receptors from Carmot Therapeutics, Inc.

The kit is an assemblage of materials or components, including at least one of the compositions described herein, comprising a dual GLP-1/GIP receptor agonist.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat, reduce the severity of, inhibit or brain insulin resistance and related disorders in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of a composition containing dual GLP-1/GIP receptor agonist, or an analog, pharmaceutical equivalent or a peptidomimetic thereof. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Effective treatments of brain insulin resistance in AD may thus be effective treatments of the disorder itself. AD might be treatable, then, with antidiabetics safe for human use that cross the blood-brain barrier. Yet only one class of such drugs consistently show promise as AD therapeutics: receptor agonists of the incretins, glucagon-like peptide-1 (GLP-1) and glucose-dependent insulinotropic peptide (GIP). These include the FDA-approved GLP-1 agonist liraglutide (Victoza), which has been studied extensively in the APP/PS1 mouse model of AD. This model develops severe brain insulin resistance by 7.5 months based on ex vivo stimulation tests. Liraglutide (25 nmol/kg) given ip daily for 2 months virtually eliminates brain insulin resistance in APP/PS1 mice. Perhaps for that reason, the same drug regimen also restores their synaptic plasticity and reduces their AD-like pathologies (Aβ plaque load, microglial activation, synaptic loss, and vascular damage) and AD-like symptoms (poor object recognition and spatial memory).

Liraglutide is likely to have the same therapeutic effects in AD cases given our findings that such cases have severely diminished brain levels of GLP-1 and that exposure of this drug (100 nM) to brain tissue from MCI and AD dementia cases for just one hour significantly reduces insulin resistance in that tissue. In MCI tissue, the drug elevates insulin responsiveness 60-75%. Even greater elevations are expected using peptides activating both GLP-1 and GIP incretin receptors, because the therapeutic effects noted with liraglutide on the AD mouse model are also exerted by GIP agonists alone. As that predicts, dual GLP-1/GIP agonists are 1.5-2.0 times more potent than liraglutide in reducing Abeta plaque loads in the mouse model.

Apart from their beneficial actions in the brain, incretin agonists offer the added benefit of treating peripheral resistance, which can exacerbate AD pathology. These agonists are also among the few promising AD therapeutics sufficiently tested in humans to meet the goal of the National Plan to Address Alzheimer's Disease to prevent and effectively treat this disorder by 2025.

For the reasons summarized herein, incretin peptide agonists, especially dual GLP-1/GIP agonists, are emerging as high priority candidates for the first effective treatment of AD.

FIG. 1 shows a diagrammatic illustration of the likely role played by brain insulin resistance in AD. As listed on the left side of the diagram, many early pathogenic factors in AD can promote the development of brain insulin resistance, which can in turn promote development of many late pathogenic features of the disorder listed on the right side of the diagram. For that reason, brain insulin resistance could accelerate AD pathogenesis. Reducing brain insulin resistance is thus expected to slow the clinical progression of the disorder.

As shown in FIGS. 2A-2B there are at least two mechanisms causing brain insulin resistance in AD. Circled numbers indicate the sequence of steps resulting in impaired neuronal insulin signaling in each mechanism. The first mechanism (FIG. 2A) is mediated by serine phosphorylation of insulin receptor substrate-1 (IRS-1). Soluble Aβ oligomers activate microglial cells (step 1), which then secrete inflammatory cytokines, among them IL-1β, IL-6, and TNFα (step 2). Via neuronal receptors, each of these cytokines activates one or more of four IRS-1 serine kinases, namely JNK, IKK, ERK, and PKR (step 3). Each of these kinases phosphorylate one of more IRS-1 sites, specifically S312, S616, and/or S636 (numbers based on human IRS-1), (step 4). Such phosphorylation, which is very high in AD dementia, impairs IRS-1 inter-actions with the IR upstream (step 5), inhibits activation of IRS-1 (step 6), and thereby IRS-1 activation of PI3K (step 7), which inhibits transmission of insulin signals from reaching down-stream targets. Inactivation of IRS-1 leads to its removal from signaling domains and its degradation (step 8). Along with reduced insulin responsiveness of the IR, steps 5-8 induce insulin resistance, which is known to promote formation of Aβ oligomers (step 9).

The second mechanism (FIG. 2B) is not driven by IRS-1 pS, but by elevated levels of leptin (step 1), which occurs in the brains of AD cases. Activation of the leptin receptor (LR) phosphorylates STAT3 (step 2), which dimerizes (step 3) and leads to transcription (step 4) of SOCS3 (suppressor of cytokine signaling 3). SOCS3 binds the insulin receptor (step 5) and disrupts its interaction with and activation of IRS-1 (steps 6 and 7), resulting in impaired down-stream insulin signaling (step 8) and loss of functional IRS-1 (step 9).

In the experiments and data described herein, the dual GLP-1/GIP receptor agonist is the peptide having the sequence YXEGTFTSDYSIYLDKQAAXEFVNWLLAGGPSSGAPPPSK, as described as peptide 18 in Finan, B. et al. [Science Translational Medicine 5: 209ra151, 2013], wherein X is aminoisobutyric acid and peptide 18 is acylated at Lys-40 ((K⁴⁰-C₁₆ acyl)-NH₂), as described as compound 19 in Finan, B. et al. Science Translational Medicine 5: 209ra151, 2013.

As shown in FIG. 3, brain insulin resistance in AD is closely associated with cognitive decline. This was discovered with biomarkers of brain insulin resistance, such as IRS-1 pS616, when studying cases for which postmortem tissue and cognitive test results were available on the same cases [Talbot, K. et al. Journal of Clinical Investigation 122: 1316-1338, 2012]. The three graphs show linear regression plots of combined data from 30 normal, 29 MCI, and 30 AD dementia cases. Each graph plots the relationship between density (cells per mm²) of neurons with elevated IRS-1 pS616 (i.e., insulin resistant neurons) in field CA1 of the hippocampus with composite scores from the same cases on multiple tests of episodic memory (FIG. 3A), working memory (FIG. 3B), or global cognition (FIG. 3C). Note the highly negative associations between density of insulin resistant neurons and cognitive status, which remained strong after adjusting for age, sex, years of education, Aβ plaque load, and neurofibrillary tangle densities. The strength of the negative associations is emphasized by the very large percentage of the variance (R²) in the memory scores accounted for by the density of neurons with elevated IRS-1 pS616.

As shown in FIG. 4, brain insulin resistance is marked in mild cognitive impairment (MCI) and in AD dementia (FIG. 4A), as well as in the APP/PS1 transgenic mouse model of AD (FIG. 4B). The graphs show responsiveness of the hippocampal formation to direct application of 1 nM insulin for 30 min at 3 levels of a key signaling pathway: (1) the insulin receptor beta chain (IRβ)→(2) insulin receptor substrate-1 (IRS-1)→(3) protein kinase B (Akt). Percent increases in activated forms of these molecules (IRβ pY960, IRS-1 pY, and Akt pS473) following insulin stimulation were used as measures of insulin responsiveness. The human data are based on study of age-, sex-, and postmortem interval-matched cases (10 cognitively normal [N], 10 non-amnestic MCI [naMCI], 10 amnestic MCI [aMCI], and 10 Alzheimer's disease dementia [ADd] cases). The mouse data are based on study of 7-month female animals: 8 wild-type (WT) and 8 transgenic (Tg) APP/PS1 mice. Asterisks indicate statistical significance of reductions in insulin responsiveness (**=p<0.001, ***=p<0.0001).

FIG. 5 shows that peripheral administration of an incretin receptor agonist (liraglutide) substantially reduces hippocampal formation insulin resistance in a transgenic mouse model of AD. Female wild-type (WT) and transgenic (Tg) APP/PS1 mice (n=8 per group) were injected ip with vehicle (saline) or a diabetic dose (25 nmoles/kg body wt.) of liraglutide daily for two months starting at 5 months of age. After removal from the skull, their brains were fresh frozen and later thawed for removal of the hippocampal formation. Ex vivo experiments were then performed to test responsiveness of the hippocampal tissue to direct application of 1 nM insulin for 30 minutes as explained in FIG. 4. Insulin responsiveness was tested at the level the IR beta chain (IRβ), IRS-1, and Akt by measuring insulin-stimulated levels of their activated forms (IRβ py960, IRS-1 pY612, and Akt pS473) compared to those forms measured in the absence of insulin. In animals treated only with saline, insulin responsiveness was markedly diminished, especially below the level of IRβ. In those treated with liraglutide, insulin responsiveness remained normal in the WT mice, but was markedly improved in the Tg mice. The asterisks (***) indicate p<0.001 for differences between Tg and WT animals on saline and for differences between Tg animals on drug versus no drug.

FIG. 6 shows that ex vivo exposure of hippocampal formation tissue from MCI and AD dementia cases to 100 nM liraglutide for 30 minutes prior to 1 nM insulin stimulation for 30 minutes significantly improves its insulin responsiveness. Tissue was obtained from age-sex, and postmortem interval-matched cases from the Alzheimer's disease Center at Rush University: 10 normal (N), 10 non-amnestic MCI (naMCI), 10 amnestic MCI (aMCI), and 10 AD dementia (ADd) cases. Insulin resistance in the MCI and ADd cases is evident in low insulin responsiveness without liraglutide pretreatment. Such pretreatment did not induce insulin hypersensitivity in normal cases, but nearly eliminated hippocampal insulin resistance in naMCI cases at all levels of the insulin signaling pathway (e.g., at the level of the IR, IRS-1, and Akt shown here) and in amnestic MCI (aMCI) cases at the level of the IR. Hippocampal insulin resistance was substantially reduced in AD dementia (ADd) cases at the IR level and in aMCI cases below the IR. Yet insulin responsiveness remained far from normal even after liraglutide pretreatment at IRS-1 and Akt levels in aMCI and especially AD dementia cases. Asterisks indicate statistical significance levels for differences in insulin responsiveness within diagnostic groups (*=p<0.05, **=p<0.01, and *** p=<0.001).

FIG. 7 shows that a dual GLP-1/GIP receptor agonist is superior to a single GLP-1 receptor agonist (liraglutide) in reducing one cause of brain insulin resistance in AD: elevated basal levels of serine phosphorylated insulin receptor substrate-1 (IRS-1 pS) (see FIG. 2A). The data shown are ratios of IRS-1 pS to total IRS-1 in ex vivo Western blotting tests on the hippocampal formation of age- and sex-matched normal (N), non-amnestic mild cognitive impairment (naMCI), amnestic mild cognitive impairment (aMCI), and AD dementia (ADd) cases (n=10 in each group). Levels of IRS-1 pS were normal in the naMCI cases, but are elevated in aMCI and ADd cases. Liraglutide significantly reduced basal IRS-1 pS in the aMCI cases, but not in the ADd cases. The dual GLP-1/GIP receptor agonist significantly reduced basal IRS-1 pS levels in both aMCI and ADd cases. The dual agonist was also more effective than liraglutide in doing this in the aMCI cases. Asterisks indicate statistical significance levels for differences within diagnostic groups (*=p<0.05, **=p<0.01, and *** p=<0.001); ns=not significantly different.

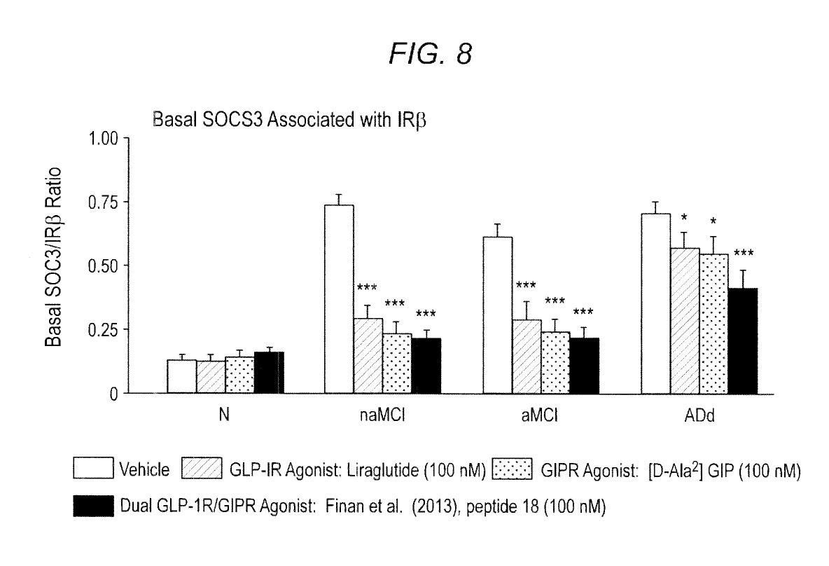

FIG. 8 shows that a dual GLP-1/GIP receptor agonist (dual IRA) is superior to single IRAs (the GLP-1 receptor agonist liraglutide or the GIP receptor agonist D-Ala² GIP) in reducing another cause of brain insulin resistance in AD: elevated basal levels of SOCS (suppressor of cytokine signaling 3) associated with the insulin receptor (see FIG. 2B). The data shown are ratios of SOCS3 to IR beta chain (IRβ) in ex vivo Western blotting tests on the hippocampal formation of age- and sex-matched normal (N), non-amnestic mild cognitive impairment (naMCI), amnestic mild cognitive impairment (aMCI), and AD dementia (ADd) cases (n=10 in each group). The basal SOCS3/IRβ levels were highly elevated in naMCI, aMCI, and ADd cases compared to the normal cases. Both single IRAs (liraglutide and [D-Ala²] GIP) and the dual IRA were highly effective in reducing this ratio in MCI cases, but the dual IRA had a significantly greater effect in ADd cases. Asterisks indicate statistical significance levels for differences within diagnostic groups (*=p<0.05 and *** p=<0.0001).

FIGS. 9A-9B shows that a dual incretin receptor agonist (IRA) is superior to single IRAs (the GLP-1 receptor agonist liraglutide or the GIP receptor agonist D-Ala² GIP) in raising insulin responsiveness of the hippocampal formation from amnestic MCI (aMCI) and AD dementia (ADd) cases. The data displayed derive from ex vivo insulin stimulation experiments explained in FIG. 4 and performed on the same cases used for FIGS. 4, 6-8. Both graphs show the effect of 30 min pretreatment of hippocampal formation tissue with single or dual IRAs on subsequent 30 min 1 nM insulin-stimulated activation of IRS-1 as measured by IRS-1 pY levels. The upper graph (FIG. 9A) shows the percent increase in IRS-1 pY following 1 nM insulin exposure above IRS-1 pY levels seen without insulin stimulation. The lower graph (FIG. 9B) shows the percent increase in insulin-induced IRS-1 pY after a given IRA versus no such pretreatment. None of the IRAs affected insulin responsiveness in normal cases. All of them substantially and equivalently raised insulin responsiveness in naMCI cases. In aMCI and especially ADd cases, the dual AD IRA was markedly more effective than either single IRA in raising insulin responsiveness toward normal levels. The dual IRA raised insulin responsiveness to a significantly greater degree (p<0.001) than the single IRAs in aMCI and ADd cases. Asterisks indicate statistical significance levels for differences within diagnostic groups (*=p<0.05, **=p<0.01, and *** p=<0.001); ns=not significantly different.

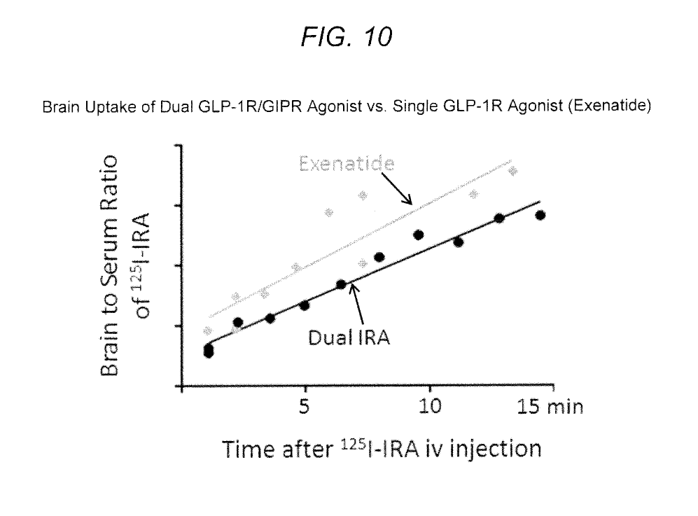

FIG. 10 shows unidirectional brain influx rate (Ki) of a dual IRA versus a single IRA (exeantide) in mice (C57Bl/6J). The Ki is 0.55±0.01 μL-min for exenatide and 0.20±0.01 μL-min for dual IRA (p<0.001 compared to brain uptake of ¹³¹I-albumin).

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method for treating, inhibiting, reducing the severity of and/or reducing progression of a disease-state in a subject in need thereof comprising:
(i) providing a dual incretin receptor agonist (IRA); and
(ii) administering a therapeutically effective amount of the composition to the subject so as to treat the disease-state in the subject.

2. The method of claim 1, wherein the disease-state is brain insulin resistance.

3. The method of claim 1, wherein the disease-state is mild cognitive impairment (MCI), Alzheimer's disease (AD) dementia, Parkinson's disease and/or traumatic brain injury.

4. The method of claim 1, wherein the incretin receptor agonist is dual GLP-1 receptor and GIP receptor (GLP-1/GIP receptor) agonist.

5. The method of claim 3, wherein the subject has diabetes.

6. The method of claim 3, wherein the subject does not have diabetes.

7. The method of any one of claim 1, wherein the composition is administered subcutaneously, intravenously, intramuscularly, intraperitonealy, orally or via inhalation.

8. The method of claim 1, wherein the effective amount of the incretin receptor agonist is about 0.1 to 0.5 mg/kg/day, 0.5 to 5 mg/kg/day, 5 to 10 mg/kg/day, 10 to 20 mg/kg/day, 20 to 50 mg/kg/day, 10 to 100 mg/kg/day, 10 to 120 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day or 900 to 1000 mg/kg/day.

9. The method of claim 1, wherein the subject is human.

10. The method of claim 1, wherein the composition is administrated to the subject before, during, or after the subject develops the disease-state.

11. The method of claim 1, wherein the composition is administrated to the subject 1-3 times per day or 1-7 times per week.

12. The method of claim 1, wherein the composition is administrated to the subject for 1-5 days, 1-5 weeks, 1-5 months, or 1-5 years.

13. The method of claim 3, further comprising administering to the subject with Alzheimer's disease dementia any one or more of cholinesterase inhibitors, tacrine (Cognex), NMDA receptor antagonist or a combination thereof.

14. The method of claim 13, wherein the cholinesterase inhibitor is donepezil (Aricept), galantamine (Razadyne) or rivastigmine (Exelon).

15. The method of claim 13, wherein the NMDA receptor antagonist is memantine (Namenda).

16. The method of claim 4, wherein the dual incretin receptor agonist comprises the sequence YXEGTFTSDYSIYLDKQAAXEFVNWLLAGGPSSGAPPPSK or YXEGTFTSDYSIYLDKQAAXEFVNWLLAGGPSSGAPPPSC, wherein X is aminoisobutyric acid.

17. The method of claim 4, wherein the dual incretin receptor agonist consists of the sequence YXEGTFTSDYSIYLDKQAAXEFVNWLLAGGPSSGAPPPSK or YXEGTFTSDYSIYLDKQAAXEFVNWLLAGGPSSGAPPPSC, wherein X is aminoisobutyric acid.

18. The method of claim 16 or 17, wherein the dual incretin receptor agonist is modified.

19. The method of claim 18, wherein modification comprises acylation or PEGylation of the peptide having the sequence YXEGTFTSDYSIYLDKQAAXEFVNWLLAGGPSSGAPPPSK or YXEGTFTSDYSIYLDKQAAXEFVNWLLAGGPSSGAPPPSC, wherein X is aminoisobutyric acid.

20. The method of claim 16 or 17, wherein the peptide YXEGTFTSDYSIYLDKQAAXEFVNWLLAGGPSSGAPPPSK is acylated at Lys (K) at position 40.
